(19) United States
(12) Patent Application Publication
(10) Pub. No.: US 2007/0148678 A1
(43) Pub. Date: Jun. 28, 2007

(54) Device and method for carrying out a nucleic acid test, and method for producing such a device

(76) Inventors: Thomas Ehben, Weisendorf (DE); Hans-Dieter Feucht, Renningen (DE); Christian Zilch, Leipzig (DE)

Correspondence Address: HARNESS, DICKEY & PIERCE, P.L.C., P.O.BOX 8910, RESTON, VA, 20195, US

(21) Appl. No.: 11/636,454

(22) Filed: Dec. 11, 2006

(30) Foreign Application Priority Data
Dec. 13, 2005 (DE) 10 2005 059 535.9

(51) Int. Cl. C12Q 1/68; C12M 1/34

(52) U.S. Cl. 435/6; 435/287.2

(57) ABSTRACT

A device and a method are disclosed for carrying out nucleic acid tests. The device includes a substrate and one or more gel pads arranged on the substrate and designed so that at least one of the biological and chemical reactions necessary for the test takes place in them, particularly a polymerase chain reaction. A method for producing such a device is also disclosed.

### PRIORITY STATEMENT

The present application hereby claims priority under 35 U.S.C. §119 on German patent application number DE 10 2005 059 535.9 filed Dec. 13, 2005, the entire contents of which is hereby incorporated herein by reference.

### FIELD

Embodiments of the invention generally relate to a device and/or a method for carrying out a nucleic acid test; for example one in which a sample is to be tested for a plurality of target sequences. Furthermore, embodiments of the invention also generally relate to a method for producing such a device.

### BACKGROUND

At present, there is generally a demand to standardize biological and chemical reaction processes for detecting a substance and integrate them as far as possible into a test strip, so that the detection tests can be carried out even by non-specialized personnel. This has already been achieved in the case of immunoassays: immunoassays are already successfully available as a fast test on convenient test strips.

For other detection reactions and assays as well, all the process steps from sample preparation to detection have also been integrated successfully on a plastic card (cartridge). The processes on such a cartridge are controlled by a reader, in which the cartridge is placed. The target molecules are detected at the end of the process with the aid of biochips (microarrays) which are equipped with specific capture molecules, and the biological information is read out optically, electrically or magnetically.

However, the integration of various preparation steps on a plastic card requires complex microfluidics on the cartridge, which need to be controlled by the reader. The production of such cartridges is therefore substantially more elaborate and expensive than the aforementioned immunoassay test strips. Furthermore, controlling the processes and the signal readout requires complex equipment, which is likewise expensive to produce.

Such test strips or cartridges are not commercially available at present for nucleic acid tests in which a sample is to be tested for the presence of different DNA or RNA sequences. The reason for this is that nucleic acid tests require complex sample preparation, amplification of the target sequences and detection with specific gene probes. Currently, these steps are almost exclusively carried out manually in the laboratory.

The fastest and simplest nucleic acid tests at present involve homogeneous assays with the aid of Peltier block PCRs or a lightcycler PCR. Such an assay will be described briefly below:

First, the nucleic acids need to be isolated from the sample and purified. To this end, for example, The DNA isolation kit from Qiagen may be used.

Amplification of the target DNA strands is subsequently carried out, for example by a PCR (polymerase chain reaction), in order to multiply the DNA contained in the sample. PCR is used in order to multiply a short, accurately defined part of a DNA strand. The reagents necessary for this are: a DNA strand which contains the segment to be multiplied, two primers for establishing the start and end of the segment to be multiplied, a thermally stable DNA polymerase for replicating the established segment, nucleotides i.e. the building blocks for the DNA strand synthesized by the polymerase, and a buffer solution for providing a suitable chemical environment.

The PCR process comprises a plurality of thermocycles, each with three steps: the double-stranded DNA contained in the sample is first heated in order to separate the strands. The temperature is then reduced so that the primers can bind to the DNA single strands. In the last step, the DNA segment between the primers is filled in by the polymerase with the respectively complementary nucleotides. This cycle is repeated about 10-50 times.

In order to detect the presence of a DNA sequence multiplied in this way, a gene probe tagged for example with a fluorescent dye is added in homogeneous assays, which hybridizes with a particular DNA segment during or after the thermocycling and thereby allows indirect detection of the intended target sequence. The fluorescence changes according to the concentration of the target sequences to be detected in the solution. The light quanta are detected with the aid of detectors, which are placed either directly in the thermocycler (lightcycler) or externally in an additional reader.

Such a homogeneous assay, however, has the disadvantage that only a limited number of different target sequences can be detected. The limitation is due to the number of primers and gene probes which can be used simultaneously in a reaction. The capacity of the thermocycler is also limited, since only a certain number of samples can be treated in parallel. The restriction in the number of gene probes generally results from the number of available fluorescent dyes, the fluorescence spectrum of which can still be recorded separately by the existing detectors and filters.

For carrying out a nucleic acid test, no fast test is therefore yet available which could be carried out by inexperienced personnel or even the actual patient at the point of care. A laboratory infrastructure with skilled personnel and the necessary equipment and materials is generally required.

GUSCHIN, D. et al., Manual manufacturing of oligonucleotide, DNA, and protein microchips. Anal. Biochem. (1997) 250 (2) 203-11 discloses a device according to the generic type for carrying out a nucleic acid test. A sample can be tested for a plurality of target sequences in this device; it has a substrate and one or more gel pads arranged on the substrate, which are designed so that at least one of the biological or chemical reactions necessary for the test takes place in them.

Other devices are disclosed in YERSHOV, G. et al., DNA analysis and diagnostics on oligonucleotide microchips. Proc. Natl. Acad. Sci. USA (1996) 93 (10) 4913-8 and in MIKHAILOVICH, V. et al., Identification of rifampin-resistant Mycobacterium tuberculosis strains by hybridization, PCR, and ligase detection reaction on oligonucleotide microchips. J. Clin. Microbiol. (2001) 39 (7) 2531-40.

### SUMMARY

In at least one embodiment of the present invention, a device is provided for carrying out a nucleic acid test, a method is provided for producing the device and/or a method is provided for carrying out a nucleic acid test. At least one example embodiment of such a device and/or such methods makes it possible to carry out a nucleic acid test less expensively with an improved workflow.

Advantageous configurations of embodiments of the device and of the method are specified hereafter. In so far as they are applicable, the features specified in the method claims may also be employed in the claimed device, and vice versa.

The device according to at least one embodiment of the invention includes a substrate and one or more gel pads, which are arranged on the substrate and are designed so that at least one of the biological or chemical reactions necessary for the test takes place in them. The substrate is advantageously a strip of plastic, board or composites thereof.

The workflow is therefore decisively simplified compared with conventional nucleic acid tests. Since no elaborate microfluidics are necessary, the test is also less susceptible to interference. A so-called “lab-on-a-strip” solution is furthermore very cost-effective compared with the conventional laboratory methods, even compared with new cartridges or “lab-on-a-chip” systems.

Depending on the size or number of the gel pads, many reactions can be carried out simultaneously on the substrate. There are therefore scarcely any limitations in respect of the number of reactions which can be carried out in parallel.

At least the detection reaction necessary for detecting the intended target sequences preferably takes place in the gel pads. In particular different gene probes, which respectively hybridize with a particular segment of the DNA, are used in the individual gel pads or in different regions of a single gel pad.

The detection of target molecules may take place in a variety of ways: on the one hand, similarly as homogeneous PCR in a liquid medium, it is possible to use fluorescence-tagged gene probes which are equipped with a quencher. When the molecule of the gene probe hybridizes onto a complementary target sequence and the quencher is removed by the exonuclease activity of the polymerase, the fluorescence can be detected by means of a fluorescence microscope or a CCD camera. The precise mechanism of the detection reaction and the generation of the fluorescent radiation etc. are known to the person skilled in the art and need not be explained in detail here. Alternatively, precipitation reactions, color reactions or the formation of molecule conglomerates in the gel pad may be recorded optically by turbidimetric, nephelometric or colorimetric methods. It is also conceivable to use radioactive tags.

Amplification of the target sequence furthermore preferably takes place in the gel pads, particularly by polymerase chain reaction (PCR). The reagents necessary for this, for example primers, polymerase, buffer solution etc., are preferably present already in the gel pad. Alternatively, however, it may be the user who first loads the device with the necessary reagents.

The gel pads are preferably formed from an aqueous phase and a gel matrix made of a hydrophilic cross-linked polymer. The gel matrix should be formulated so that DNA molecules from the sample can penetrate into the gel bed, and so that freedom of movement by diffusion is provided inside the gel bed, in order to ensure acquisition by the polymerase. In order to carry out a PCR, the gel matrix should furthermore be sufficiently heat-stable and not disintegrate at temperatures of up to 100° C.

In particular transversely cross-linked polymers based on polyacrylamide, polyacrylic acid, polyhydroxyethyl methacrylate, polyvinyl alcohol and polyvinyl pyrrolidone are suitable for the construction of such a gel matrix. Additional linker group-containing comonomers, for example glycidyl methacrylate or maleic acid imide, are advantageously also polymerized into the gel matrix. These additional monomers may serve both to improve the adhesion on the gel matrix of the substrate and for covalently coupling probe molecules into the gel pad.

The substrate is preferably formed by a plastic strip, although it may also be formed by coated card, board or a composite of plastic and board. If a plurality of gel pads are applied onto the substrate, then they are preferably delimited from one another by partition walls or a well or compartment structure in the substrate. The substrate surface is preferably formulated so that the gel pads adhere well thereon. Preferably, the substrate is rectangular and is provided with an array of from 1×2 to 100×100, particularly preferably from 10×10 to 20×20 gel pads.

In order to prevent desiccation, the gel pads may be coverable with a film or placed in a pressure chamber, in order to prevent water vapor from escaping and desiccation.

At least one embodiment of the invention also relates to a method for producing such a device, which includes the following steps: providing a substrate in the form of a strip of plastic, board or composites thereof for receiving one or more gel pads, producing a mixture of the reagents necessary for the desired biological or chemical reaction and a monomer preparation for producing a cross-linked gel matrix, applying the mixture onto the substrate, and inducing a poly-reaction of the monomer preparation in order to form one or more gel pads on the substrate. The monomer preparation in this case preferably contains—besides water or an aqueous solution—at least one monomer type for producing a hydrophilic polymer chain and at least one monomer type for crosslinking these chains. The poly-reaction for gelling takes place for example by polymerization, polycondensation or polyaddition. The gelling is, for example, induced by chemical catalysts (for example radical formers) or a light reaction (for example UV irradiation).

In another embodiment of the production method, the gel pads are first produced from a monomer preparation and not loaded with the other reagents (for example primers, gel probes, polymerase, buffer) until a second step. In this case, the unladen gel pads may also be produced by photolithographic methods with the use of a mask. The loading may take place specifically by so-called spotting.

Different gel pads preferably contain different primers, so that the individual gel primers are configured for the amplification of different nucleic acid sequences. Correspondingly, various gel pads preferably also receive different gene probes for detecting different target sequences.

Lastly, at least one embodiment of the invention also concerns a method for carrying out a nucleic acid test, which includes the following steps: preparing the sample by disintegrating the cells, isolating the DNA from the cells and/or dividing the DNA into smaller segments; applying the prepared sample onto the gel pads of a device as described above; and detecting the target sequences by detecting tagged gene probes in the individual gel pads. The sample is preferably applied onto the gel pad by means of a suitable swab instrument, for example a swab stick with a cotton bud.

In at least one embodiment of the method, care should be taken that the nucleic acids to be detected must be prepared from the sample before cycling in the scope of a PCR. In this case, it is necessary to ensure that nucleic acid fragments penetrate into the gel pad in order to function as a template for a PCR. To this end, two variants are conceivable:

The DNA preparation from an arbitrary sample takes place separately with the aid of a conventional isolation Method (for example the Qiagen DNA isolation kit). The DNA is subsequently fragmented mechanically, chemically or enzymatically, before it is applied onto the gel bed by means of a suitable swab instrument (for example swab sticks with a cotton bud).

Alternatively, it is conceivable that the DNA does not need to be isolated from the sample beforehand. Before application onto the gel pad, the sample is treated with a suitable lysis solution and, after neutralization, it is applied uniformly onto the gel pad using the swab instrument. The lysis solution may, for example, contain DNAses or restriction endonucleases in a defined concentration, which cause the genomic DNA to be divided into smaller fragments and thus facilitate subsequent diffusion into the gel bed.

In an alternative embodiment the DNA is transported actively into the gel pad, for example by electrophoresis. In this embodiment two electrodes are therefore arranged above and below, or on two sides of the gel pad, in order to generate an electric field. The electrodes, i.e. at least the lower electrode, may be integrated into the substrate.

### BRIEF DESCRIPTION OF THE DRAWINGS

The invention will now be explained in more detail with the aid of example embodiments with reference to the appended drawings. In the drawings:

FIG. 1 shows a cross section through a test strip according to a first embodiment of the invention;

FIG. 2 shows a cross section through a test strip according to a second embodiment of the invention;

FIG. 3 shows a cross section through a test strip according to a third embodiment of the invention;

FIG. 4 shows a plan view of a test strip with a swab instrument;

FIG. 5 shows a cross section through a test strip in a pressure chamber;

FIG. 6 shows a cross section through a test strip with an evaluation device according to a first embodiment;

FIG. 7 shows a cross section through a test strip with an evaluation device according to a second embodiment;

FIG. 8 shows a cross section through a test strip according to yet another embodiment of the invention.

### DETAILED DESCRIPTION OF THE EXAMPLE EMBODIMENTS

The terminology used herein is for the purpose of describing particular embodiments only and is not intended to be limiting of the present invention. As used herein, the singular forms “a”, “an” and “the” are intended to include the plural forms as well, unless the context clearly indicates otherwise. It will be further understood that the terms “includes” and/or “including”, when used in this specification, specify the presence of stated features, integers, steps, operations, elements, and/or components, but do not preclude the presence or addition of one or more other features, integers, steps, operations, elements, components, and/or groups thereof.

In describing example embodiments illustrated in the drawings, specific terminology is employed for the sake of clarity. However, the disclosure of this patent specification is not intended to be limited to the specific terminology so selected and it is to be understood that each specific element includes all technical equivalents that operate in a similar manner.

Referencing the drawings, wherein like reference numerals designate identical or corresponding parts throughout the several views, example embodiments of the present patent application are hereafter described.

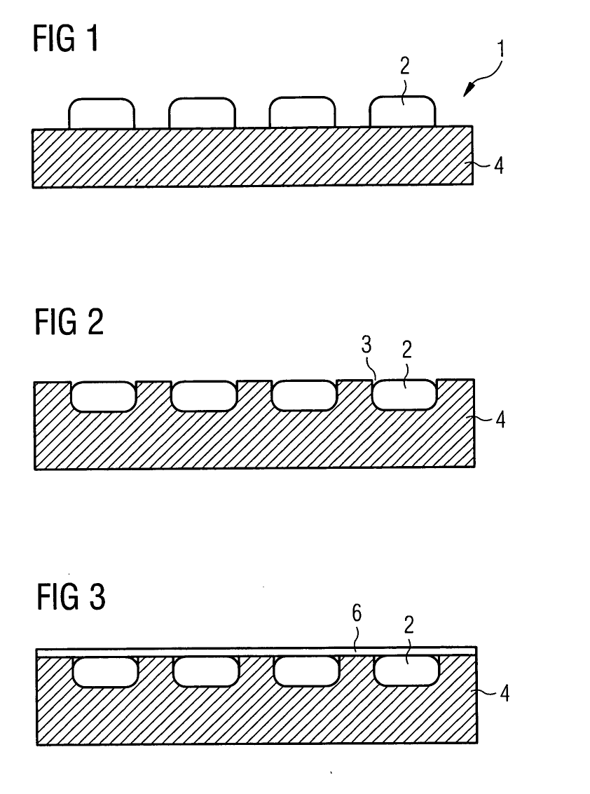

FIG. 1 shows a device for carrying out a nucleic acid test 1, also referred to below as a test strip, in a cross section. The test strip essentially comprises a substrate 4 and gel pads 2 arranged thereon. The substrate or strip 4 consists, for example, of plastic or board with a surface suitable for sufficient adhesion of the pads. These are preferably applied by spotting in the ungelled state. Alternatively, the gel pads may also be produced on a different surface and transferred in the gelled state onto the test strip 4. Instead of a gel, it is in principle possible to use any other suitable carrier medium with a suitable viscosity, which is as high as possible.

In FIGS. 2 and 3, the substrate 4 is equipped with suitable wells 3 so that the reagents present in the individual gel pads do not mix together. The delimitation also facilitates subsequent readout of the results. Alternatively, a plurality of different reactions may also take place in a single gel pad, particularly when they use at least partially the same reagents.

The test strip of FIG. 3 is additionally covered with a plastic film 6, which may preferably be removed temporarily in order to apply the sample. Instead of the film 6 it is also possible to use any form of lid, albeit one which is preferably transmissive for the fluorescent radiation of the tags used.

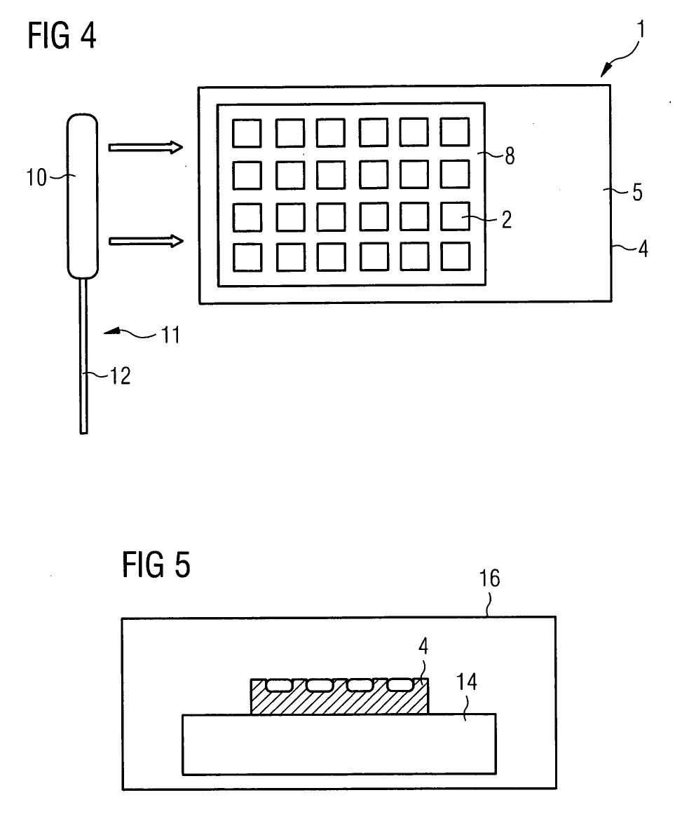

FIG. 4 shows a test strip 1 in plan view. The substrate 4 includes a region 5 which is left free in order to hold the strip, and an array region 8 which comprises a matricial arrangement of a plurality of gel pads 2. The sample is preferably applied using the swab instrument 11 which comprises a cotton bud 10, adapted to the width of the test strip 4, and a handle 12. Alternatively, the sample may also be applied by pipetting.

The test strip is preferably placed on an appropriate heating element in order to carry out the thermocycling necessary for the PCR. This is respectively denoted by 14 in FIGS. 5-7. It may, for example, be a Peltier element or a hot-air chamber. Standard equipment may be used, although it is also possible to use a thermoelement adapted to the size and thickness of the test strip. Owing to the small size of the test strip, the thermocyclers required are less expensive than in the case of conventional assays.

In the examples shown in FIGS. 5-7, the test strip 1 and the thermoelement 14 are respectively arranged in a pressure chamber which prevents water vapor from escaping and desiccation.

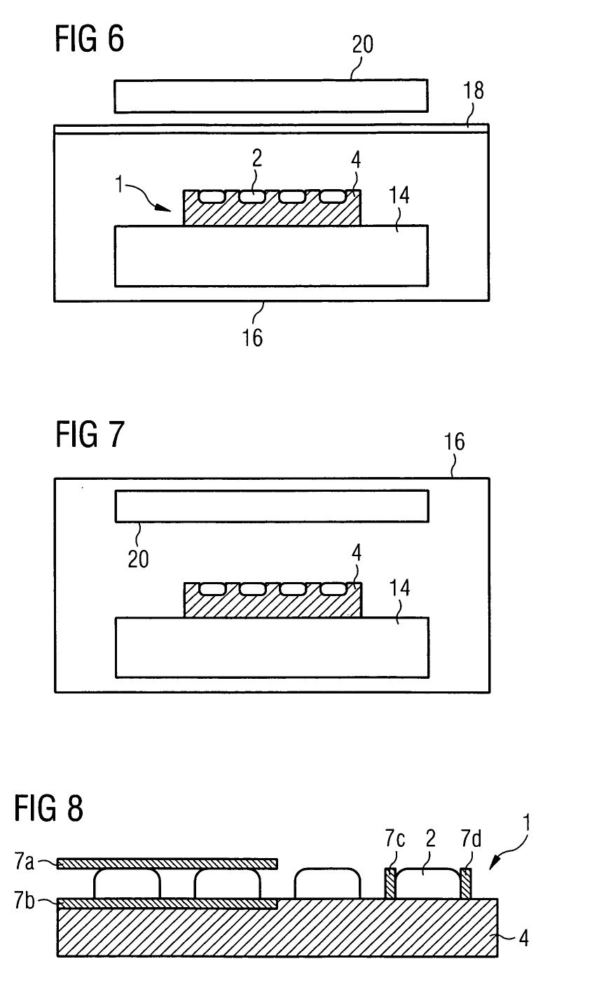

According to FIGS. 6 and 7, a reader 20 is preferably also provided which detects the detection reaction taking place in the individual gel pads. This may be a fluorescence microscope, a CCD camera or a gamma camera. The evaluation unit 20 may be arranged outside the pressure chamber according to FIG. 6. In the case of an optical evaluation unit, a transparent lid 18 is then used for the pressure chamber. Alternatively, as shown in FIG. 7, the evaluation unit 20 may be integrated into the pressure chamber 16.

FIG. 8 shows an example embodiment of a test strip which allows active transport of the DNA into the gel pads by electrophoresis. To this end, two options are shown for the arrangement of corresponding electrodes 7a to 7d in order to generate an electric field: according to the left-hand embodiment, the lower electrode 7b may be embedded in the substrate 4 or rest on it. The gel pads 2, on which the second electrode 7a bears, are arranged on the lower electrode 7b. A strong electric field extending in the vertical direction can be generated using this arrangement, by which the DNA molecules migrate into the gel pad. The right-hand side of the test strip shows the preferred embodiment, in which the electrodes 7c and 7d are respectively arranged on the left and right of a gel pad 2. In a gel pad array, even more electrodes may sometimes be provided.

Embodiments of the invention make it possible to perform homogeneous reactions in a parallel multiplex method, which can be carried out extremely cost-effectively on a substrate strip having gel pads.

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A device for carrying out a nucleic acid test, in which a sample is to be tested for a plurality of target sequences, comprising:
a substrate in the form of a strip of at least one of plastic, board and composites of plastic or board; and
one or more gel pads, arranged on the substrate and designed so that at least one of biological and chemical reactions necessary for the test takes place in the one or more gel pads.

2. The device as claimed in claim 1, wherein at least a detection reaction necessary for detecting intended target sequences, particularly a hybridization of the target sequences with gene probes, takes place in the one or more gel pads.

3. The device as claimed in claim 1, wherein an amplification of the target sequences takes place in the one or more gel pads.

4. The device as claimed in claim 1, wherein the reagents necessary for the at least one of biological and chemical reactions are contained in the one or more gel pads.

5. The device as claimed in claim 1, wherein the one or more gel pads contain a gel matrix made of a hydrophilic cross-linked polymer, the polymer being selected from the group consisting of polyacrylamide, polyacrylic acid, polyhydroxyethyl methacrylate, polyvinyl alcohol and polyvinyl pyrrolidone.

6. The device as claimed in claim 5, wherein further comonomers which contain linker groups are polymerized into the gel matrix.

7. The device as claimed in claim 1, wherein the one or more gel pads are delimited from one another by at least one of partition walls and a well structure in the substrate.

8. The device as claimed in claim 2, wherein the one or more gel pads include at least two gel pads, wherein each of the gel pads respectively contain different gene probes which are tagged with at least one of radioactive isotopes, fluorescent dyes and enzymes, and wherein the device further comprises a measuring instrument to detect the tag in each individual gel pad.

9. The device as claimed in claim 1, wherein the one or more gel pads are covered with a film.

10. A method for producing a device for carrying out a nucleic acid test, in which a sample is to be tested for a plurality of target sequences, comprising:
providing a substrate in the form of a strip of at least one of plastic, board and composites of plastic or board, for receiving one or more gel pads;
producing a mixture of reagents necessary for a desired at least one of biological and chemical reaction and a monomer preparation for producing a cross-linked gel matrix;
applying the mixture onto the substrate; and
inducing a poly-reaction of the monomer preparation in order to form the one or more gel pads on the substrate.

11. A method for producing a device for carrying out a nucleic acid test, in which a sample is to be tested for a plurality of target sequences, comprising:
providing a substrate in the form of a strip of at least one of plastic, board and composites of plastic or board, for receiving one or more gel pads;
producing a monomer preparation for producing a cross-linked gel matrix;
applying the preparation onto the substrate;
inducing a poly-reaction of the monomer preparation in order to form the one or more gel pads on the substrate; and
loading the one or more gel pad or pads with a mixture of reagents necessary for a desired at least one of biological and chemical reaction.

12. The method as claimed in claim 10, wherein the one or more gel pads are designed so that a PCR takes place in them, and wherein the mixture contains the primers necessary for a PCR, a thermally stable polymerase and a suitable buffer solution.

13. The method as claimed in claim 12, wherein a plurality of gel pads are formed on the substrate, and wherein the mixture for different gel pads contains different primers so that the gel pads are configured for the amplification of different nucleic acid sequences.

14. A method for carrying out a nucleic acid test, in which a sample is to be tested for a plurality of target sequences, comprising:
preparing the sample by at least one of disintegrating the cells, isolating DNA from the cells and dividing the DNA into smaller segments;
applying the prepared sample onto gel pads of a device including a substrate in the form of a strip of at least one of plastic, board and composites of plastic or board and the gel pads; and
detecting the target sequences by detecting tagged gene probes in the individual gel pads.

15. The method as claimed in claim 14, wherein the sample is applied onto the gel pads via a suitable swab instrument.

16. The method as claimed in claim 14, wherein the DNA migrates into the gel pads by electrophoresis.

17. The device as claimed in claim 2, wherein at least a detection reaction necessary for detecting a hybridization of the target sequences with gene probes, takes place in the one or more gel pads.

18. The device as claimed in claim 2, wherein an amplification of the target sequences takes place in the one or more gel pads.

19. The device as claimed in claim 3, wherein an amplification of the target sequences by polymerase chain reaction (PCR), takes place in the one or more gel pads.

20. The device as claimed in claim 5, wherein the gel matrix includes at least one of glycidyl methacrylate and maleic acid imide.

21. The method as claimed in claim 11, wherein the one or more gel pads are designed so that a PCR takes place in them, and wherein the mixture contains the primers necessary for a PCR, a thermally stable polymerase and a suitable buffer solution.

22. The method as claimed in claim 21, wherein a plurality of gel pads are formed on the substrate, and wherein the mixture for different gel pads contains different primers so that the gel pads are configured for the amplification of different nucleic acid sequences.
